(19) United States
(12) Patent Application Publication
(10) Pub. No.: US 2023/0321077 A1
(43) Pub. Date: Oct. 12, 2023

(54) Combination treatment of dilated cardiomyopathy

(71) Applicant: GreenStone Biosciences, Inc., Palo Alto, CA (US)

(72) Inventors: Joseph C. Wu, Palo Alto, CA (US); Chun Liu, Palo Alto, CA (US); Christopher David Yan, San Francisco, CA (US); Celine Lai, Fremont, CA (US)

(73) Assignee: GreenStone Biosciences, Inc., Palo Alto, CA (US)

(21) Appl. No.: 18/132,919

(22) Filed: Apr. 10, 2023

Related U.S. Application Data
(60) Provisional application No. 63/329,809, filed on Apr. 11, 2022.

(51) Int. Cl. A61K 31/4709; A61K 31/366; A61P 9/00

(52) U.S. Cl. CPC A61K 31/4709; A61P 9/00; A61K 31/366

(57) ABSTRACT

Treatment of dilated cardiomyopathy in a subject by combination treatment with a tyrosine kinase inhibitor and a statin.

### CROSS-REFERENCE TO RELATED APPLICATION

This application claims the benefit under 35 USC 119(e) of Application No. 63/329,809, filed 11 Apr. 2022, the entire content of which is incorporated into this application by reference.

### BACKGROUND OF THE INVENTION

### Field of the Invention

This invention relates to the combination treatment of dilated cardiomyopathy.

### Description of the Related Art

Dilated Cardiomyopathy

Cardiomyopathies are diseases of the heart muscle that render the heart unable to properly pump enough blood to the body. In the dilated form of cardiomyopathy (called dilated cardiomyopathy or DCM), the heart is enlarged. As the heart enlarges, it becomes less effective in pumping blood, which then leads to symptoms of heart failure and irregular heart rhythms (arrhythmias). DCM is principally characterized by left ventricular enlargement and/or a reduction in systolic function, more precisely described as a reduction in left ventricular ejection fraction less than <40% or fractional shortening less than 25% (Hershberger et al., “Clinical and genetic issues in dilated cardiomyopathy: a review for genetics professionals”, Genet. Med., 12, 655-667 (2010)). It is estimated that approximately 1 out of every 2500 persons has DCM, although the disease is probably even more common. DCM affects both men and women and can affect both adults and children. As with other types of cardiomyopathies, DCM is a chronic disease without a known cure. However, the treatments currently available can significantly improve its course.

There are many possible causes of dilatation and dysfunction of the heart, such as coronary artery disease, infection, and excessive use of alcohol. In cases where the cause of DCM is unknown, the condition is called “idiopathic” dilated cardiomyopathy. About one-third to one-half of patients with idiopathic DCM have a family history of the disease in one or more relatives. These patients are considered to have familial dilated cardiomyopathy. Familial DCM is caused by defective genes that affect the function of the heart muscle. Several familial DCM genes are currently known, whereas others are still under investigation.

DCM may involve mutations in one or more of the following genes: LMNA, FLNC, BAG3, DES, MYH7, PLN, RBM20, SCN5A, TNNCI, TNNT2, TTN, DSP, ACTC1, ACTN2, JPH2, NEXN, TNNI3, TPM1, VCL, ABCC9, ANKRD1, CSRP3, CTF1, DSG2, DTNA, EYA4, GATAD1, ILK, LAMA4, LDB3, MYBPC3, MYH6, MYL2, MYPN, NEBL, NKX2-5, OBSCN, PLEKHM2, PRDM16, PSEN2, SGCD, TBX20, TCAP, TNNI3K, LRRC10, NPPA, MIB1, MYL3, PDLIM3, PKP2, PSEN1, and others. These genes can contribute to familial DCM, which can result in abnormally elevated platelet-derived growth factors (PDGF). In idiopathic DCM, and particularly in familial DCM, a pathogenic variant of the lamin A (LMNA) or filamin C (FLNC) gene may be the underlying cause for DCM (Hershberger et al.; Brayson et al., “Current insights into LMNA cardiomyopathies: Existing models and missing LINCs”, Nucleus, 8, 17-33 (2017).

LMNA is a gene that encodes the intermediate filament proteins, lamin A and C, which localize between the nuclear membrane and the chromatin. Mutations in the LMNA gene lead to disruption of cellular functions, which can result in a milieu of diseases referred to as laminopathies (Brayson et al.; Lu et al., “LMNA cardiomyopathy: cell biology and genetics meet clinical medicine”, Dis. Model Mech., 4, 562-568 (2011)). Laminopathies all share a degree of nuclear fragility, altered nuclear architecture, impaired nuclear signaling and transcriptional activation through alterations in adaptive or protective mechanisms. LMNA is one of few established genes that has a clear genotype to clinical phenotype relationship, which has proven to be associated with conduction defects, malignant ventricular arrhythmias, and supraventricular arrhythmias preceding the development of left ventricular dilation and heart failure DCM (Hershberger et al.).

Unlike other cases of familial DCM, often the first sign of LMNA-related DCM is sudden cardiac arrest leading to death, owing to prevalence of arrhythmias, e.g., ventricular tachycardia and/or ventricular fibrillation (Lee et al., “Activation of PDGF pathway links LMNA mutation to dilated cardiomyopathy”, Nature, 572, 335-340 (2019)).

Specific modifications of lamin A that have been associated with DCM include Q6*, R26G, K32del, A57P, L59R, R60G, E82K, L85R, K117fs, R133P, L140R, E145K, E161K, N195K, E203G, H222P, R225Q, R249Q, Y267C, R298C, D300G, E358K, M371K, R377H, R453W, R527P, T528R, L530P, R541G, R571S, S573L, V607V, G608S, G608G, and Q656Q.

Filamin C, encoded by the FLNC gene, is an isoform of the filamin family, predominantly expressed in skeletal and cardiac muscle (Razinia et al., “Filamins in mechanosensing and signaling”, Ann. Rev. Biophys., 41, 227-246 (2012); Zhou et al., “Filamins in cell signaling, transcription and organ development”, Trends Cell Biol., 20, 113-123 (2010)). Filamin C modulates cell stiffness by regulating cross-linking of actin filaments and anchorage of the actin-cytoskeleton to transmembrane proteins at cell-cell adhesion sites. Because of its interactions with transmembrane and Z-disc proteins, mutations in filamin C would be expected to dysregulate intracellular signaling. Mutations in filamin C have been associated with myofibrillar skeletal myopathies, hypertrophic cardiomyopathy, restrictive cardiomyopathy, DCM, and arrhythmogenic cardiomyopathy (Brodehl et al., “Mutations in FLNC are Associated with Familial Restrictive Cardiomyopathy”, Hum. Mutat., 37, 269-279 (2016); Tucker et al., “Novel Mutation in FLNC (Filamin C) Causes Familial Restrictive Cardiomyopathy”, Circ. Cardiovasc. Genet., 10 (2017); Valdes-Mas et al., “Mutations in filamin C cause a new form of familial hypertrophic cardiomyopathy”. Nat. Commun., 5, 5326 (2014)).

Carriers of filamin C truncating mutations are at high risk of developing dilated and arrhythmogenic cardiomyopathies (Begay et al., “FLNC Gene Splice Mutations Cause Dilated Cardiomyopathy”, JACC Basic Transl. Sci., 1, 344-359 (2016); Begay et al., “Filamin C Truncation Mutations Are Associated With Arrhythmogenic Dilated Cardiomyopathy and Changes in the Cell-Cell Adhesion Structures”, JACC Clin. Electrophysiol., 4, 504-514 (2018)). Specific modifications of filamin C that have been associated with DCM include Y83*, F106L, R991*, P963R, A1183L, A1186V, V1198Gfs*64, S1642L, G1891Vfs61X, G2011E, 12160F, E2189X, V2297M, P2298L, G2299S, R2318W, R2410C, and Y2563C.

The platelet-derived growth factor receptors (PDGFRs) are transmembrane proteins belonging to the receptor tyrosine kinase class (Kanaan et al., “Use of multitarget tyrosine kinase inhibitors to attenuate platelet-derived growth factor signalling in lung disease”, Eur. Respir. Rev., 26: 170061 (2017)). Components of the PDGF signaling pathway are upregulated during the early phases of cardiomyocyte differentiation but become downregulated in fully differentiated cardiomyocytes (Chintalgattu et al., “Cardiomyocyte PDGFRβ signaling is an essential component of the mouse cardiac response to load-induced stress”, J. Clin. Invest., 120, 472-484 (2010); Lee et al.). Previous studies have shown that expression and activation of PDGFRβ dramatically increases in response to pressure overload-induced stress as is seen in hypertension, which has been associated with other clinically used tyrosine kinase/PDGFR inhibitors (Chintalgattu et al.; Lee et al.). This suggests that, under normal physiological conditions, the PDGF signaling pathway in adult cardiomyocytes is present but not active. However, abnormal PDGFRB and PDGFRA activation has been implicated in the pathogenesis of cardiomyopathy induced by LMNA mutations and FLNC mutations (Chen et al., (2022). “Activation of PDGFRA signaling contributes to filamin C-related arrhythmogenic cardiomyopathy”, Sci. Adv., 8, eabk0052; Lee et al.). Treatment with tyrosine kinase/PDGFR inhibitors can partially reverse the pathological gene expression profile, cardiac dysfunction, and electrical instability in patient cardiomyocytes carrying LMNA or FLNC mutations.

Tyrosine Kinase Inhibitors

Tyrosine kinase inhibitors have revolutionized the management and outcomes of chronic myeloid leukemia patients (Pasvolsky et al., “Tyrosine kinase inhibitor associated vascular toxicity in chronic myeloid leukemia”, Cardiooncology, 1, 5 (2015)). However, over the past few years, evidence of varying quality has emerged demonstrating an association between treatment with TKIs and vascular toxicity (Valent et al., “Vascular safety issues in CML patients treated with BCR/ABL1 kinase inhibitors”, Blood, 125, 901-906 (2015)). The main vascular adverse events recognized thus far have been peripheral arterial occlusive disease (PAOD), although others include cerebral ischemia, myocardial infarction and pulmonary hypertension (Manouchehri et al., “Tyrosine Kinase Inhibitors in Leukemia and Cardiovascular Events: From Mechanism to Patient Care”, Arterioscler. Thromb. Vasc. Biol., 40, 301-308 (2020); Pasvolsky et al.). Nilotinib was found to upregulate proatherogenic adhesion proteins on human endothelial cells, including ICAM1 (intercellular adhesion molecule 1), VCAM1 (vascular cell adhesion protein 1), and E-selectin, which collectively can recruit inflammatory cells and platelets and promote vascular events (Sukegawa et al., “The BCR/ABL tyrosine kinase inhibitor, nilotinib, stimulates expression of IL-1beta in vascular endothelium in association with downregulation of miR-3p”, Leuk. Res/, 58, 83-90 (2017)). In addition, a recent series of elegant experiments suggest that ponatinib vascular toxicity involves platelet adhesion followed by secondary microvascular angiopathy (Latifi et al., “Thrombotic microangiopathy as a cause of cardiovascular toxicity from the BCR-ABL1 tyrosine kinase inhibitor ponatinib”, Blood, 133, 1597-1606 (2019)). Studies have been performed to determine the mechanisms of pulmonary hypertension associated with dasatinib (Guignabert et al., “Dasatinib induces lung vascular toxicity and predisposes to pulmonary hypertension”, J. Clin. Invest., 126, 3207-3218 (2016)).

Representative tyrosine kinase inhibitors include axitinib, cediranib, crenolanib, dasatinib, imatinib, linifenib, motesanib, nilotinib, pazopanib, ponatinib, quizartinib, sorafenib, and sunitinib.

Statins

HMG-CoA reductase (3-hydroxy-3-methyl-glutaryl-coenzyme A reductase) inhibitors, commonly referred to as statins, are a class of lipid-lowering medications that reduce illness and mortality in those who are at high risk of cardiovascular disease. They are the most common cholesterol-lowering drugs. Statins are known to generally improve vascular function in disease conditions (Wolfrum et al., “Endothelium-dependent effects of statins”, Arterioscler. Thromb. Vasc. Biol., 23, 729-736 (2003)). Moreover, a subset of statins can ameliorate endothelial dysfunction by restoring downregulated KLF2 expression in vitro as well as improving the vascular reactive hyperemia index in vivo (Sayed et al., “Clinical trial in a dish using iPSCs shows lovastatin improves endothelial dysfunction and cellular cross-talk in LMNA cardiomyopathy”, Sci. Transl. Med., 12, eaax9276 (2017)). Representative statins include atorvastatin, fluvastatin, lovastatin, pitavastatin, pravastatin, rosuvastatin, and simvastatin.

The disclosures of the documents referred to in this application are incorporated into this application by reference.

### SUMMARY OF THE INVENTION

This invention is a method of treating dilated cardiomyopathy in a subject by concomitant administration of a tyrosine kinase inhibitor and a statin.

In other aspects, this invention includes:
pharmaceutical compositions for treating dilated cardiomyopathy comprising: a tyrosine kinase inhibitor and a statin; and
kits for treating dilated cardiomyopathy comprising: (a) compositions comprising a tyrosine kinase inhibitor, and (b) compositions comprising a statin.

Because concomitant administration of a tyrosine kinase inhibitor and a statin has shown activity against dilated cardiomyopathy in two assays, the concomitant administration of a tyrosine kinase inhibitor and a statin is expected to show efficacy in the treatment of dilated cardiomyopathy.

Subjects may be tested for the presence of a mutation in the subject's LMNA or FLNC gene, or lamin A or filamin C protein, before treatment.

Preferred embodiments of this invention are characterized by the specification and by the features of Claims 1 to 10 of this application as filed.

### DETAILED DESCRIPTION OF THE INVENTION

### Definitions

“Dilated cardiomyopathy” (DCM) is described in the section entitled “Dilated cardiomyopathy” in the DESCRIPTION OF THE RELATED ART.

“Tyrosine kinase inhibitors” are described in the section entitled “Tyrosine kinase inhibitors” in the DESCRIPTION OF THE RELATED ART.

“Statins” are described in the section entitled “Statins” in the DESCRIPTION OF THE RELATED ART.

Salts (for example, pharmaceutically acceptable salts) of tyrosine kinases and of statins are included in this invention and are useful in the methods described in this application. These salts are preferably formed with pharmaceutically acceptable acids or bases, as appropriate. See, for example, “Handbook of Pharmaceutically Acceptable Salts”, Stahl and Wermuth, eds., Verlag Helvetica Chimica Acta, Zürich, Switzerland, for an extensive discussion of pharmaceutical salts, their selection, preparation, and use. Unless the context requires otherwise, any reference to tyrosine kinase inhibitors or to each of the tyrosine kinase inhibitors, such as imatinib, to statins or to each of the statins, such as atorvastatin, is a reference both to the compound and to its salts. The tyrosine kinase inhibitors generally form, and are supplied as, acid addition salts, typically with organic acids (e.g., imatinib is generally supplied as imatinib mesylate and sunitinib as sunitinib malate); while the statins generally form, and are supplied as, salts with alkali metal or alkaline earth bases (e.g., atorvastatin is generally supplied as atorvastatin calcium and fluvastatin as fluvastatin sodium), or are supplied as the non-salt compound (e.g., lovastatin and simvastatin are generally supplied as the non-salt compounds).

“Concomitant administration” of a tyrosine kinase inhibitor and a statin means administration of the tyrosine kinase inhibitor and the statin during the course of treatment of dilated cardiomyopathy. This concomitant administration may involve administration of the tyrosine kinase inhibitor before, during, and/or after administration of the statin, such that therapeutically effective levels of each of the compounds are maintained. Concomitant administration may be accomplished by administration of the tyrosine kinase inhibitor and the statin each at its usual dosing; but if both are orally bioavailable and conveniently have daily oral dosing, concomitant administration might include also administration of a combination dosage form. “Combination therapy” with a tyrosine kinase inhibitor and a statin has the same meaning as “concomitant administration”.

A “therapeutically effective amount” of a tyrosine kinase inhibitor, or of a statin, means that amount of each which, when the tyrosine kinase inhibitor and the statin are concomitantly administered to a human for treating dilated cardiomyopathy, is sufficient to effect treatment for the dilated cardiomyopathy.

“Treating” or “treatment” of dilated cardiomyopathy, in a human includes one or more of:
(1) preventing or reducing the risk of developing dilated cardiomyopathy, i.e., causing the clinical symptoms of dilated cardiomyopathy not to develop in a subject who may be predisposed to dilated cardiomyopathy but who does not yet experience or display symptoms of the dilated cardiomyopathy (i.e., prophylaxis);
(2) inhibiting dilated cardiomyopathy, i.e., arresting or reducing the development of dilated cardiomyopathy or its clinical symptoms; and
(3) relieving dilated cardiomyopathy, i.e., causing regression, reversal, or amelioration of the dilated cardiomyopathy or reducing the number, frequency, duration or severity of its clinical symptoms. The therapeutically effective amount for a particular subject varies depending upon the health and physical condition of the subject to be treated, the extent of the dilated cardiomyopathy, the assessment of the medical situation, and other relevant factors. It is expected that the therapeutically effective amount will fall in a relatively broad range that can be determined through routine trial.

“Treatment” does not necessarily imply “cure” or complete treatment, e.g., treatment of all clinical symptoms of dilated cardiomyopathy, though “treatment” may include “cure”. Rather, “treatment” implies the provision of clinical benefit by the administration of the combination of the tyrosine kinase inhibitor and the statin when compared to administration of none or only one of the tyrosine kinase inhibitor and the statin; and treatment may also be assessed by improvement in biological markers of the dilated cardiomyopathy being treated.

The therapeutically effective amount for a particular subject varies depending upon the health and physical condition of the subject to be treated, the extent of the dilated cardiomyopathy, the assessment of the medical situation, and other relevant factors. It is expected that the therapeutically effective amount will fall in a relatively broad range that can be determined through routine trial.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

Subjects Suitable for Treatment by this Invention

Suitable subjects for treatment by the method, composition, kit, etc. of this invention are humans suffering from dilated cardiomyopathy, such as subjects suffering from idiopathic dilated cardiomyopathy or familial dilated cardiomyopathy. In particular, suitable subjects include those determined to have mutation in one or both of their LMNA and FLNC genes, leading to mutations in their lamin A and/or filamin C proteins, as mentioned in the section entitled “Dilated cardiomyopathy” in the DESCRIPTION OF THE RELATED ART; and testing for the presence of mutated LMNA and/or FLNC genes, or lamin A and/or filamin C proteins, may be appropriate and lead to treatment being given to those most likely to benefit from it.

Formulation and Administration

The tyrosine kinase inhibitor and the statin may be administered by any route suitable to the subject being treated and the nature of the subject's condition. Routes of administration include oral administration (generally preferred, if available); administration by injection, including intravenous, intraperitoneal, intramuscular, and subcutaneous injection; by transmucosal (e.g., intranasal, buccal, sublingual, rectal, or vaginal) or transdermal (topical) delivery; and the like. Formulations may be oral formulations (e.g., tablets, capsules, or oral solutions or suspensions); injectable formulations (e.g., solutions); and formulations designed to administer the drug across mucosal membranes or transdermally. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because both the tyrosine kinase inhibitors and the statins are generally orally available, typical formulations will be oral, and typical dosage forms will be tablets or capsules for oral administration (potentially including both the tyrosine kinase inhibitor and the statin in a combination dosage form). Intravenous formulations may be particularly applicable for administration to acutely ill subjects, such as those subjects who may be hospitalized for treatment.

Depending on the intended mode of administration, the pharmaceutical compositions may be in the form of solid, semi-solid or liquid dosage forms, preferably in unit dosage form suitable for single administration of a precise dosage. In addition to an effective amount of the tyrosine kinase inhibitor and/or the statin, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional pharmaceutically acceptable excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Typically, a pharmaceutical composition of the tyrosine kinase inhibitor or of the statin is packaged in a container with a label, or instructions, or both, indicating use of the pharmaceutical composition in the treatment of dilated cardiomyopathy. Typically, a pharmaceutical composition of the combination of the tyrosine kinase inhibitor and the statin, or a kit comprising separate compositions of the tyrosine kinase inhibitor and the statin, is packaged in a container with a label, or instructions, or both, indicating use of the pharmaceutical composition or kit in the treatment of dilated cardiomyopathy.

A suitable amount of the tyrosine kinase inhibitor for oral dosing when administered in combination with the statin (subjects with dilated cardiomyopathy may well be taking other therapies in addition to the tyrosine kinase inhibitor and the statin discussed in this application) is expected to be 1-25 mg/day. That is, a suitable amount of the tyrosine kinase inhibitor for oral dosing is expected to be similar to the amounts employed in current clinical practice for renal cell carcinoma treatment at the lower end when used alone.

A suitable amount of the statin for oral dosing when administered in combination with the tyrosine kinase inhibitor (subjects with dilated cardiomyopathy may well be taking other therapies in addition to the tyrosine kinase inhibitor and the statin discussed in this application) is expected to be 1-100 mg/day, such as 5-80 mg/day, depending on the relative potency of the statin being used (see, for example, the Wikipedia entry on statins, https://en.wikipedia.org/wiki/Statin, for a table of equivalent dosages when used for cholesterol lowering). That is, a suitable amount of a statin for oral dosing is expected to be similar to the amounts employed in current clinical practice for cholesterol lowering when used alone.

However, it is possible that the therapeutically effective amounts of either may be less when used in combination therapy than when either is administered alone.

A person of ordinary skill in the art of the treatment of dilated cardiomyopathy will be able to ascertain a therapeutically effective amount of the tyrosine kinase inhibitor and the statin, when used by concomitant administration, for a particular subject and stage of dilated cardiomyopathy, to achieve a therapeutically effective amount without undue experimentation and in reliance upon personal knowledge and the disclosure of this application.

### EXAMPLES

### Example 1 (Pre-Clinical, Single Agent, In Vitro)

Induced pluripotent stem cells (iPSCs) carrying the K117fs mutation in lamin A, from a patient with dilated cardiomyopathy, were differentiated into cardiomyocytes (iPSC-CMs) and endothelial cells (iPSC-ECs) respectively.

iPSC-CMs carrying the K117fs mutation in lamin A exhibited a large percentage of arrhythmic cells; and these cells were treated with the tyrosine kinase inhibitor sunitinib to inhibit the arrhythmic phenotype. At day 28 after differentiation, test iPSC-CMs were treated with sunitinib, dissolved in dimethyl sulfoxide (DMSO), at a concentration of 500 nM; while control cells were treated with an equal concentration of DMSO; for 48 hours. Prior to arrhythmia recording, test and control iPSC-CMs were separately enzymatically dissociated and plated on 48-well plates (Axion Biosystems) that previously had been coated with ESC-qualified Matrigel (Corning). The arrythmia recordings were performed using Microelectrode Array technology (MEA) from Axion Biosystems, at 37° C. in a 5% CO₂ environment. Data acquisition was performed using Axion Biosystem software, and data analysis was completed using the CIPA software. Treatment with sunitinib at 500 nM ameliorated the arrhythmic phenotype of lamin A K117fs iPSC-CMs: percentage of arrhythmic cells 27% with sunitinib treatment, 72.5% with DMSO control.

iPSC-ECs carrying the K117fs mutation in lamin A showed downregulated expression of the KLF2 gene; and these cells were treated with the statin lovastatin to restore KLF2 gene expression. Test iPSC-ECs were treated with lovastatin at a concentration of 1 μM; while control cells were untreated. The KLF2 gene expression in the cells was performed using quantitative real-time (RT)-PCR. Total RNA was extracted using the RNeasy plus mini Kit (Qiagen) according to the manufacturer's instructions. cDNA was generated using qScript cDNA SuperMix (Quantabio). Quantitative RT-PCR was conducted on the StepOne Real-Time PCR system (Life Technologies) using TaqMan Universal PCR Master Mix (Life Technologies), and RT-qPCR probes from Life Technologies. All samples were normalized against GAPDH as relative expression measurement.

Treatment with lovastatin at 1 μM restored the KLF2 expression of lamin A K117fs iPSC-ECs to 4.5-fold that of untreated cells.

### Example 2 (Pre-Clinical, Single Agent and Combination, In Vitro)

iPSC-ECs are cultured with DMSO, sunitinib (S), crenolanib (C), lovastatin (L), and the combinations of sunitinib+lovastatin (S+L) and crenolanib+lovastatin (C+L), generally as in Example 1. The restoration of KLF2 expression of the iPSC-ECs and the repression of SELE (selectin E, a pro-atherogenic gene induced by tyrosine kinase inhibitors in vascular toxicity) are similarly measured, also as in Example 1. The results are as follows:

| Reagent | KLF2 gene expression | SELE gene expression |
|---|---|---|
| DMSO | 1.03 ± 0.11 | 1.04 ± 0.15 |
| S | 1.01 ± 0.14 | 1.58 ± 0.41 |
| C | 0.73 ± 0.05 | 0.60 ± 0.09 |
| L | 1.29 ± 0.05 | 1.11 ± 0.22 |
| S + L | 1.31 ± 0.07 | 1.25 ± 0.18 |
| C + L | 0.64 ± 0.04 | 0.82 ± 0.11 |

An examination of the cell morphology showed that sunitinib caused dramatic cell morphology changes (probably through migration/proliferation) and that lovastatin could rescue the morphology.

Similar results are seen with other tyrosine kinase inhibitors and other statins; and similar results are seen with cells containing other lamin A mutations and/or filamin C mutations.

### Example 2 (Preclinical, Combination, LMNA^{H2222P/H2222P} Mouse)

The LMNA^{H2222P/H2222P} mouse was first described by Arimura et al., “Mouse model carrying H222P-LMNA mutation develops muscular dystrophy and dilated cardiomyopathy similar to human striated muscle laminopathies”, Hum. Mol. Genetics, 14(1), 155-169 (2005) and is now well known as a model for human LMNA dilated cardiomyopathy (see, for example, Wu et al., “Mitogen-Activated Protein Kinase Inhibitors Improve Heart Function and Prevent Fibrosis in Cardiomyopathy Caused by Mutation in Lamin A/C Gene”, Circulation, 123, 53-61 (2011)). LMNA^{H2222P/H2222P} mice, 16 weeks old and therefore already showing symptoms of cardiomyopathy, are treated with placebo (for example, the injection vehicle), a tyrosine kinase inhibitor, such as crenolanib, and a statin, such as simvastatin, alone and in combination, by i.p. injection of the compounds at 3 mg/Kg/day; and assessed for cardiomyopathy by left ventricle dilatation and ejection fraction (measured by echocardiography) after four weeks of treatment. Treatment with the combination restores greater function than with placebo or either agent alone.

### Example 3 (Clinical, Combination)

Patients with dilated cardiomyopathy, especially those with one or more of a lamin A mutation and/or a filamin C mutation, are treated with therapeutically effective amounts of a tyrosine kinase inhibitor, such as crenolanib, and a statin, such as simvastatin. The patients show an improvement in their condition as manifested by a reduction in their symptoms, such as reductions in tiredness, leg swelling, shortness of breath, and chest pain; and improvements in cardiac function as measured by electrocardiogram, echocardiogram, and chest X-ray.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

What is claimed is:

1. A method of treating dilated cardiomyopathy in a subject, comprising concomitant administration to the subject of a therapeutically effective amount of:
(a) a tyrosine kinase inhibitor; and
(b) a statin.

2. The method of claim 1 where the tyrosine kinase inhibitor is axitinib, cediranib, crenolanib, dasatinib, imatinib, linifenib, motesanib, nilotinib, pazopanib, ponatinib, quizartinib, sorafenib, or sunitinib.

3. The method of claim 1 or 2 where the statin is atorvastatin, fluvastatin, lovastatin, pitavastatin, pravastatin, rosuvastatin, or simvastatin.

4. The method of claim 1 where the subject has a mutation in either or both of the subject's lamin A and filamin C proteins.

5. The method of claim 4 where the subject has a mutation in the subject's lamin A protein.

6. The method of claim 4 where the subject has a mutation in the subject's filamin C protein.

7. The method of claim 4 where the subject has a mutation in both the subject's lamin A and filamin C proteins.

8. The method of claim 4 where the mutation in the subject's lamin A protein is one or more of Q6*, R26G, K32del, A57P, L59R, R60G, E82K, L85R, K117fs, R133P, L140R, E145K, E161K, N195K, E203G, H222P, R225Q, R249Q, Y267C, R298C, D300G, E358K, M371K, R377H, R453W, R527P, T528R, L530P, R541G, R571S, S573L, V607V, G608S, G608G, and Q656Q.

9. The method of claim 4 where the mutation in the subject's filamin C protein is one or more of Y83*, F106L, R991*, P963R, A1183L, A1186V, V1198Gfs*64, S1642L, G1891Vfs61X, G2011E, 12160F, E2189X, V2297M, P2298L, G2299S, R2318W, R2410C, and Y2563C.

10. The method of claim 1 where the subject is tested for the presence of a mutation in the subject's LMNA or FLNC gene, or lamin A or filamin C protein, before treatment.

11. The method of claim 2 where the subject has a mutation in either or both of the subject's lamin A and filamin C proteins.

12. The method of claim 3 where the subject has a mutation in either or both of the subject's lamin A and filamin C proteins.

13. The method of claim 2 where the subject is tested for the presence of a mutation in the subject's LMNA or FLNC gene, or lamin A or filamin C protein, before treatment.

14. The method of claim 3 where the subject is tested for the presence of a mutation in the subject's LMNA or FLNC gene, or lamin A or filamin C protein, before treatment.
